(19) United States
(12) Patent Application Publication
(10) Pub. No.: US 2003/0109577 A1
(43) Pub. Date: Jun. 12, 2003

(54) Pharmaceutical composition containing citalopram

(76) Inventors: Ken Liljegren, Vaerlose (DK); Per Holm, Vanlose (DK); Ole Nielsen, Valby (DK); Sven Wagner, Sodertalje (SE)

Correspondence Address: DARBY & DARBY P.C., 805 Third Avenue, New York, NY, 10022, US

(21) Appl. No.: 09/730,380

(22) Filed: Dec. 5, 2000

(30) Foreign Application Priority Data
Oct. 27, 2000 (DK) PA 2000 01614

(51) Int. Cl. C07D 307/78; C07D 307/87; C07D 307/93; A61K 31/34; A01N 43/08

(52) U.S. Cl. 514/469; 549/467

(57) ABSTRACT

A solid unit dosage form comprising citalopram, which is prepared by direct compression of a mixture of citalopram base or a pharmaceutically acceptable salt thereof and pharmaceutically acceptable excipients, or by filling of said mixture in a hard gelatine capsule. Large crystals of a pharmaceutical acceptable salt of citalopram and method for the manufacture of said large crystals.

### BACKGROUND OF THE INVENTION

The present invention relates to a novel pharmaceutical composition containing citalopram, 1-[3-(dimethylamino)propyl)]-1-(4-fluorophenyl)-1,3-dihydro-5-isobenzo-furancarbonitrile.

Citalopram is a well-known antidepressant drug that has the following structure:

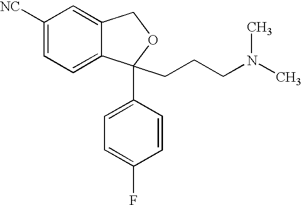

It is a selective, centrally active serotonin (5-hydroxytryptamine; 5-HT) reuptake inhibitor, accordingly having antidepressant activities.

Citalopram was first disclosed in DE 2,657,013, corresponding to US. Pat. No. 4,136,193. This patent publication describes their preparation of citalopram by one method and outlines a further method, which may be used, for preparing citalopram. The citalopram prepared was isolated in crystalline form as the oxalate, the hydrobromide and the hydrochloride salt, respectively. Furthermore, the citalopram base was obtained as an oil (B.P. 175 C/0.03 mmHg). The publication also outlines the manufacture of tablets containing salts of citalopram. Citalopram is marketed as the hydrobromide and the hydrochloride, respectively,

Manufacture of crystalline citalopram base is disclosed in co-pending DK 2000 00402. This patent publication describes the preparation of crystalline citalopram base and the use of crystalline citalopram base as an intermediate in the purification of crude citalopram hydrobromide into pure citalopram hydrobromide. The publication also outlines the manufacture of tablets containing citalopram base.

Citalopram is marketed in a number of countries as a tablet prepared by compression of granulated citalopram hydrobromide, lactose and other excipients.

It is well recognized that preparation of tablets with a reproducible composition requires that all the dry ingredients have good flow properties. In cases, where the active ingredient has good flow properties, tablets can be prepared by direct compression of the ingredients. However, in many cases the particle size of the active substance is small, the active substance is cohesive or has poor flow properties.

Further, active substances with a small particle size mixed with excipients having a larger particle size will typically segregate or de-mix during the tabletting process.

The problem of small particle size and poor flowability, is conventionally solved by enlarging the particle size of the active substance, usually by granulation of the active ingredient either alone or in combination with a filler and/or other conventional tablet ingredients.

One such granulation method is the “wet” granulation process. Using this method, the dry solids (active ingredients, filler, binder etc.) are blended and moistened with water or another wetting agent (e.g. an alcohol) and agglomerates or granules are built up of the moistened solids. Wet massing is continued until a desired homogenous particle size has been achieved whereupon the granulated product is dried.

An alternative to the “wet” granulation method is the “melt” granulation, which is also known as the “thermal plastic” granulation process, where a low melting solid is used as the granulation agent. Initially, the dry solids are blended and heated until the binder melts. As the binder is liquefied and spreads over the surface of the particles, the particles will adhere to each other and form granules. The binder solidifies upon cooling forming a dry granular product.

Wet granulation as well as melt granulation are energy intensive unit operations requiring complicated and expensive equipment as well as technical skill.

The process used for the preparation of citalopram hydrobromide results in a product with a very small particle size around 2-20 μm that, as many other particulate products with a small particle size, has very poor flow properties. Thus, in order to achieve appropriate dosing of the citalopram during tabletting, it was considered necessary to make a granulate of citalopram with larger particle size and improved flow properties.

The citalopram tablet that is marketed is a tablet made from granulated citalopram hydrobromide with various excipients.

In view of the fact that direct compression is much simpler and cheaper than the processes involving granulation there is a desire for a process for direct compression of citalopram hydrobromide.

The obstacles that hitherto have hindered direct compression of citalopram tablets have now been circumvented after extensive laboratory research.

It has been found that larger particles, i.e. particles of a size comparable to the size of the filler, may be prepared by a new and inventive crystallisation process and that these particles are useful for the manufacture of directly compressed tablets. Accurate dosing in capsules may also be with such large particles.

It has also been found, that tablets with surprisingly small variation in the content of citalopram may be prepared by direct compression of citalopram hydrobromide having a significantly smaller particle size than the filler. Accurate dosing in capsules may also be achieved despite the small particle size of citalopram.

### Objects of the Invention

It is the object of the present invention to provide a novel pharmaceutical unit dosage form containing citalopram with a suitable large particle size, wherein said unit dosage form may be prepared by direct compression.

A second object of the invention is to provide a capsule containing citalopram.

A third object of the invention is to provide large crystals of a pharmaceutically acceptable salt of citalopram suitable for use in direct compression.

A fourth object of the invention is to provide a method for manufacture of large crystals of a pharmaceutically acceptable salt of citalopram.

### Summary of the Invention

The invention then, inter alia, comprises the following alone or in combination:

A solid unit dosage form comprising citalopram prepared by direct compression of a mixture of citalopram base or a pharmaceutically acceptable salt thereof and pharmaceutically acceptable excipients, or by filling of said mixture in a hard gelatine capsule.

Crystals of a pharmaceutically acceptable salt of citalopram suitable for use in a solid unit dosage form with a median particle size of at Least 40 μm.

A method for the manufacture of crystals of a pharmaceutically acceptable salt of citalopram having a median particle size of at least 40 μm and suitable for use in a solid unit dosage form wherein a solution of a pharmaceutically acceptable salt of citalopram in a suitable solvent system at a first temperature is first cooled down to a second temperature then seeded by addition of crystals of said citalopram salt followed by a holding time at said second temperature and a controlled cooling down to a third temperature whereupon said crystals are isolated by conventional solid/liquid separation techniques.

The direct compression of citalopram, a filler and other pharmaceutically acceptable excipients into tablets has the great advantage, That the granulation and a drying step is avoided. Further, as the granulation step is avoided, it is no longer necessary to add a binding agent.

As used herein, “direct compression” means that the solid unit dosage form is prepared by compression of a simple mixture of the active ingredient and excipients, without the active ingredient having been subjected to an intermediate granulation process in order to embed it in a larger particle and improve its fluidity properties.

As used herein, “binder” means an agent, which is used in wet or melt granulation processes and acts as a binder in the granulated product.

As used herein, “particle size distribution” means the distribution of equivalent spherical diameters as determined by laser diffraction at 1 bar dispersive pressure in a Sympatec Helos equipment. “Median particle size”, correspondingly, means the median of said particle size distribution.

As used herein, “refluxing temperature” means the temperature at which the solvent or solvent system refluxes or boils at atmospheric pressure.

Thus in one embodiment of the invention, the present invention relates to a tablet prepared by direct compression of a mixture of citalopram base or a pharmaceutically acceptable salt thereof and pharmaceutically acceptable excipients.

In another embodiment, the present invention relates to a capsule prepared by filling a mixture of citalopram base or a pharmaceutically acceptable salt thereof and pharmaceutically acceptable excipients in a hard gelatine capsule.

In one embodiment, the present invention relates to a solid unit dosage form comprising citalopram in crystals with a median particle size below 20 ,μm

In another embodiment, tile present invention relates to a solid unit dosage form comprising citalopram in crystals with a median particle size of at least 40 μm, preferably in the range of 40-200 μm, even more preferred 45-150 μm and most preferred 50-100 μm

Flow, segregation and demixing properties and, hence, the suitability of the citalopram crystals for direct compression depend, besides the median particle size, on the particle side distribution.

Preferably, the solid unit dosage forms according to the invention do not contain a binder.

The solid unit dosage form according to the invention may contain 2-60% w/w active ingredient calculated as citalopram base, preferably 10-40% w/w active ingredient calculated as citalopram base, and more preferred 15-25% w/w active ingredient calculated as citalopram base. Suitably, the solid unit dosage form of the invention contains 20% w/w active ingredient calculated as citalopram base.

In particular, the present invention relates to a solid unit dosage form wherein the active ingredient is citalopram hydrobromide, or citalopram hydrochloride. Preferably the active ingredient contained in the solid unit dosage form of the invention is citalopram hydrobromide.

The solid unit dosage form according to the invention may contain a filler selected from lactose, or other sugars e.g. sorbitol, mannitol, dextrose and sucrose, calcium phosphates (dibasic, tribasic, hydrous and anhydrous), starch, modified starches, microcrystalline cellulose, calcium sulphate and/or calcium carbonate, In a preferred embodiment, the solid unit dosage form of the invention does not contain lactose.

Suitably the filler is a microcrystalline cellulose such as ProSolv SMCC90 manufactured by Penwest Pharmaceuticals or Avicel PH 200 manufactured by FMC Corporation.

Besides the active ingredient and filler, the solid pharmaceutical unit dosage forms may include various other conventional excipients such as disintegrants, and optionally minor amounts of lubricants, colorants, and sweeteners.

Lubricants used according to the invention may suitably be one or more of the following metallic stearates (magnesium, calcium, sodium), stearic acid, wax, hydrogenated vegetable oil, talc and colloidal silica.

Suitably the lubricant is magnesium stearate or calcium stearate

Disintegrants include sodium starch glycolate, croscarmellose, crospovidone, low substituted hydroxypropylcellulose, modified cornstarch, pregelatizined starch and natural starch.

The solid, pharmaceutical unit dosage form of the invention may be prepared by conventional methods using a tablet press with forced feed capability.

The filled, hard gelatine capsule of the invention may be prepared by conventional methods using a capsule filler suitable for powder filling.

In one embodiment of the present invention the crystals of a pharmaceutically acceptable salt of citalopram have a median particle size in the range of 40-200 μmm, preferably 45-150 μm and even more preferred 50-120 μm.

In a preferred embodiment of the present invention the crystals are of citalopram hydrobromide or citalopram hydrochloride, preferably citalopram hydrobromide.

In yet another embodiment of the present invention crystals of a pharmaceutically acceptable salt of citalopram having a median particle size of at least 40 μm and suitable for use in a solid unit dosage form are crystallised from a solution of a pharmaceutically acceptable salt of citalopram in a suitable solvent system. Said solvent system may comprise one or more alcohols and optionally water, preferably the solvent system is a mixture of methanol and water, wherein the methanol:water weight ratio preferably is in the range of 5:1 to 50:1; even more preferred 10:1 to 30:1 and most preferred 15:1 to 25:1. Said pharmaceutically acceptable salt of citalopram is preferably dissolved in the solvent system at a temperature in the range between 50° C. and the refluxing temperature of the solvent system, preferably between 60° C. and the refluxing temperature and more,preferred between 64° C. and the refluxing temperature. The amounts of pharmaceutically acceptable salt of citalopram and solvent used are preferably corresponding to a solvent. solute weight ratio in the range of 0.5:1 to 5:1, more preferred 0.7:1 to 2:1 and most preferred 0.9:1 to 1.5:1. The solution of a pharmaceutically acceptable salt of citalopram is cooled down to a temperature, the seeding temperature, in the range of 20-40° C., preferably 25-35° C., whereupon it is seeded with citalopram crystals and kept at said seeding temperature for a holding time for crystal growth in the range of 30 minutes to 7 days, preferably I hour to 4 days and more preferred 12 to 36 hours. After said holding time, the crystallisation batch is gradually cooled down in a controlled way from the seeding temperature to the temperature at which the crystals will be isolated from the mother liquor wherein said gradual cooling down is done over a time span in the range of 5 minutes to 6 hours, preferably 15 minutes to 4 hours and more preferred 30 minutes to 2 hours. The crystals of said pharmaceutically acceptable salt of citalopram are preferably isolated from the mother liquor at a temperature in the range of (20° C., more preferred 5-15° C., using conventional separation techniques, e.g, filtration.

The small crystals of a pharmaceutically acceptable salt of citalopram used in one embodiment of the invention may be produced according to methods described in US. Pat. No. 4,136,193.

The crystals of citalopram base used in one embodiment of the invention may be produced according to methods described in co-pending DK 2000 00402.

In the following, the invention is illustrated by way of examples. However, the examples are merely intended to illustrate the invention and should not be construed as limiting.

### EXAMPLE 1

### Crystallisation of Citalopram Hydrobromide Into Large Crystals Citalopram hydrobromide (200 g) is dissolved in a mixture of methanol (200 g) and water (20 g) at 69° C. The solution is cooled down to 30 °C., seeded with citalopram hydrobromide crystals and kept at 30° C for 24 hours, whereupon it is cooled down to 10° C. within 1 hour. The crystals are isolated by filtration, washed with cold methanol and dried. The particle size distribution for the resulting crystals is listed in table 1.

### EXAMPLE 2

### Crystallisation of Citalopram Hydrobromide Into Large Crystals

Citalopram hydrobromide (12,0 kg) is dissolved in a mixture of methanol (12.5 kg) and water (1.2 kg) at reflux. The solution is cooled down to 30° C., seeded with citalopram hydrobromide crystals (27 g) and kept at 30° C. for 16 hours, whereupon it is cooled down to 10° C. within 1 hour. The crystals are isolated by filtration, washed with cold (10° C.) methanol (3.5 kg) and dried. The particle size distribution for the resulting crystals is listed in table 1.

### EXAMPLE 3

### Crystallisation of Citalopram Hydrobromide Into Small Crystals

Citalopram hydrobromide (200 kg) is dissolved in a mixture of methanol (170 L) and acetone (680 L) at 56° C. The solution is cooled down to 15° C., seeded with citalopram hydrobromide crystals (50 g), hexane (1600 L) is gradually added within 60 minutes, whereupon the suspension is left standing with moderate stirring and cooling for 8 hours. The crystals are isolated by filtration, washed, first with a cold (10° C.) mixture of acetone (50 L) and hexane than with cold (10° C.) hexane (220 L) and dried. The particle size distribution for the resulting crystals is listed in table 1.

### EXAMPLE 4

### Crystallisation of Citalopram As The Free Base

Citalopram hydrobromide (101 g) is suspended in water (500 mL) and toluene (500 mL). NaOH (60 mL, 5 N (aq)) is added and the mixture (pH>10) is stirred for 15 min before the phases are separated. The organic phase is washed with water (2 ×100 mL) and filtered through a pad of filter help. The volatiles are removed in vacuo and the title compound is obtained as an oil. n-Heptane (400 mL) is added and the mixture is heated to 70° C. On cooling, crystals forms. The white crystals of citalopram base are filtered off and dried at ambient temperature over night in vacuo.

TABLE 1
Particle size distribution (Sympatec Helos) for citalopram
hydrobromide crystals and ProSolv SCMC9O

| Quantile | Example 1 | Example 2 | Example 3 | ProSolv SCMC90 |
|---|---|---|---|---|
| (%) | (μm) | (μm) | (μm) | (μm) |
| 95 | 465.43 | 549.42 | 96.96 | 272.94 |
| 90 | 342.89 | 352.23 | 72.27 | 231.66 |
| 50 | 96.87 | 52.70 | 14.04 | 114.17 |
| 10 | 16.54 | 11.97 | 1.19 | 32.10 |
| 5 | 8.23 | 6.67 | 0.82 | 20.56 |

### EXAMPLE 5

### Tablet Prepared By Direct Compression of Small Citalopram Hydrobromide Crystals

Tablet ingredients:

| | | |
|---|---|---|
| Citalopram, HBr | 5800 g | (20% w/w) |
| ProSolv SMCC90 | 23055 g | (79.5% w/w) |
| Magnesium stearate | 145 g | (0.5% w/w) |

Citalopram hydrobromide crystals from example 3 and ProSolv SMCC90 were blended at 7 rpm for 10 min in a 100 litre Bohle PTM 200 mixer. Magnesium stearate was added and blending continued for 3 min.

25 kg of the resulting mixture was tabletted (125.000 tablets/hour) on a 30 station Fette P 1200/IC tablet press fitted with oblong, embossed, scored 5,5 ×8 mm punches. Tablet core weight was set to 125 mg. The nominal yield was 200.000 tablets. The tablet press was run until the mixture level was just above the forced feeder, i.e. the tabletting was continued as long as possible in order to identify possible segregation tendencies in the last quantities of mixture,

Tablet Properties:

Diametrical crushing strength: 70 N

Disintegration time: 30 seconds

Friability: NA

Weight variation: 0.84% relative standard deviation (measured on 20 tablets)

Punch adhesion: None observed

Citalopram Content in the Composition During Compression

Tablets were sampled throughout the compression in order to measure segregation tendency. Since there is a significant size difference between the active ingredient, citalopram hydrobromide, and the insert filler, ProSolv SMCC90, as seen in table 1, it would be expected that the unequally sized components would segregate, i.e. de-mix, during transfer from blending vessel to tablet press hopper or sitting in the tablet press hopper during tabletting.

Sampling was performed 50 times at regular intervals during tabletting, corresponding to sampling at every 4000 tablets produced. Two tablets were withdrawn for each sample.

The tablets were assayed by a validated method using UV-absorption in an aqueous solution, thus analyzing in total 100 tablets. The relative standard deviation in citalopram content was 1.6%

The variability in tablet strength is surprisingly low in view of the small particle size of citalopram hydrobromide as compared to the inert filler.

One possible explanation for this surprising and beneficial result may be that the tendency to segregation between small citalopram crystals and larger filler particles is uniquely balanced by the poor flow properties of the small crystals.

### EXAMPLE 6

### Tablet Prepared By Direct Compression of Large Citalopram Hydrobromide Crystals

Tablet ingredients:

| | |
|---|---|
| Citalopram, HBr | (20% w/w) |
| ProSolv SMCC90 | (79.5% w/w) |
| Magnesium stearate | (0.5% w/w) |

Citalopram hydrobromide crystals from example 2 and ProSolv SMCC90 were blended. Magnesian stearate was added and blending continued.

Tablets (125 mg nominel weight) were produced.

The tablets had satisfactory technical properties.

### EXAMPLE 6

### Tablet Prepared by Direct Compression of Citalopram Crystals

Tablet ingredients:

| | |
|---|---|
| Citalopram base | (16% w/w) |
| ProSolv SMCC90 | (83.3% w/w) |
| Magnesiwn stearate | (0.7% w/w) |

Citalopram base crystals from example 4 were sieved through sieve aperture of 0.3 mm and mixed with ProSaiv SMCC90 for 3 minutes in a Turbula mixer. Magnesium stearate was added and blending continued for 30 seconds.

Tablets were produced on a single punch tabletting machine Korsch EK0.

Tablet Properties:

Tablet strength, mg, 20

Nominel tablet weight, mg: 125

Tablet diameter, mm; 7

Tablet shape: Film coating, special doomed

Diametrical crushing strength. 61.6 N

Disintegration time, min: <1

Friability: 0.1%

Mean tablet weight: 125.4

Weight variation: 0.22% relative standard deviation

The tablets produced had satisfactory technical properties.

1. A solid unit dosage form comprising citalopram, characterised in that it is prepared by direct compression of a mixture of citalopram base or a pharmaceutically acceptable salt and pharmaceutically acceptable excipients, or by filling of said mixture in a hard gelatine capsule.

2. The solid unit dosage form according to claim 1, characterised in that it is a tablet prepared by direct compression of a mixture of citalopram base or a pharmaceutically acceptable salt thereof and pharmaceutically acceptable excipients

3. The solid unit dosage form according to claim 1, characterised in that it is prepared by filling a mixture of citalopram base or a pharmaceutically acceptable salt thereof and pharmaceutically acceptable excipients in a hard gelatine capsule.

4. The solid unit dosage form according to claims 1-3, characterised in that it does not contain a binder.

5. The solid unit dosage form according to claims 1-4, characterised in that it contains 2-60% w/w active ingredient calculated as citalopram base, preferably 10- 40% w/w active ingredient calculated as citalopram base and more preferred 15-25% w/w active ingredient calculated as citalopram base.

6. The solid unit dosage form according to claims 1-5, characterised in that it contains a filler selected from lactose, sugars, preferably sorbitol, mannitol, dextrose, and/or sucrose, calcium phosphates, preferably dibasic, tribasic, hydrous and/or anhydrous, starch, modified starches, microcrystalline cellulose, calcium sulfate, and/or calcium carbonate.

7. The solid unit dosage form according to claim 6, characterised in that the filler is a microcrystalline cellulose, such as ProSolv SMCC90 or Avicel PH 200.

8. The solid unit dosage form according to claims 1-7, characterised in that it contains a lubricant selected from metallic stearates (magnesium, calcium, sodium), stearic acid, wax, hydrogenated vegetable oil, talc and colloidal silica.

9. The solid unit dosage form according to claim 8, characterised in that the lubricant is magnesium stearate or calcium stearate.

10. The solid unit dosage form according to claims 1-9, characterised in that it is substantially free of lactose.

11. The solid Unit dosage form according to claim 1-10, characterised in that the active ingredient is citalopram base.

12. The solid unit dosage form according to claims 1-10, characterised in that the active ingredient is citalopram hydrobromide or citalopram hydrochloride.

13. The solid unit dosage form according to claim 12, characterised in that the active ingredient is citalopram hydrobromide.

14. The solid unit dosage form according to claims 12-13, characterised in that the active ingredient is in the form of crystals with a median particle size below 20 μm

15. The solid unit dosage form according to claims 12-13, characterised in that the active ingredient is in the form of crystals with a median particle size of at least 40 μ preferably in the range of 40-200 μm, even more preferred 45- 150 μm and most preferred 50-100 μm.

16. Crystals of a pharmaceutically acceptable salt of citalopram suitable for use in a solid unit dosage form according to claim 15, characterised in that the median particle size of the crystals is at least 40 μm.

17. Crystals according to claim 16, characterised in that the crystals are of citalopram hydrobromide or citalopram hydrochloride.

18. Crystals according to claim 17, characterised in that the crystals are of citalopram hydrobromide.

19. Crystals according to claims 16-18, characterised in that the median particle size of the crystals is in the range of 40-200 μm, preferably 45-150 μm and even more preferred 50-120 μm

20. Method for manufacture of crystals of a pharmaceutically acceptable salt of citalopram having a median particle size of at least 40 μm and suitable for use in a solid unit dosage form according to claim 15, characterised in that a solution of a pharmaceutically acceptable salt of citalopram in a suitable solvent system at a first temperature is first cooled down to a second temperature then seeded by addition of crystals of said citalopram salt followed by a holding time at said second temperature and a controlled cooling down to a third temperature whereupon said crystals are isolated by conventional solid/liquid separation techniques.

21. The method according to claim 20, characterised in that the median particle size of the crystals is in the range of 40-200 μm, preferably 45- 150 μm and even more preferred 50-120 μm.

22. The method according to claims 20-21, characterised in that the dissolved substance is citalopram hydrobromide or citalopram hydrochloride.

23. The method according to claim 22, characterised in that the dissolved substance is citalopram hydrobromide.

24. The method according to claims 20-23, characterised in that the solvent system comprises one or more alcohols and optionally water.

25. The method according to claim, 24, characterised in that the solvent system is a mixture of methanol and water.

26. The method according to claim 25, characterised in that the methanol:water weight ratio is in the range of 5:1 to 50:1; preferably 10:1 to 30:1 and more preferred 15:1 to 25:1.

27. The method according to claims 20-26, characterised in that the solvent:solute weight ratio is in the range of 0.5:l to 5:1, preferably 0.7:1 to 2:1 and more preferred 0.9:1 to 1.5:1.

28. The method according to claims 20-27, characterised in that said first temperature is in the range between 50° C. and the refluxing temperature of the solvent system, preferably between 60° C. and the refluxing temperature and more preferred between 64° C. and the refluxing temperature.

29. The method according to claims 20-28, characterised in that said second temperature is in the range of 20-40° C, preferably 25-35° C.

30. The method according to claim 20-29, characterised in that said holding time is in the range of 30 minutes to 7 days, preferably 1 hour to 4 days and more preferred 12 to 36 hours.

31. The method according to claim 20-30, characterised in that said third temperature is in the range of 0-20° C., preferably 5-15°.

32. The method according to claim 20-31, characterised in that said controlled cooling down is a gradual cooling down over a time span in the range of 5 minutes to 6 hours, preferably 15 minutes to 4 hours and more preferred 30 minutes to 2 hours.

33. The method according to claims 20-32, characterised in that said isolation of the crystals of a pharmaceutically acceptable salt of citalopram from the mother liquor is performed by filtration.
